(19) United States
(12) Patent Application Publication
(10) Pub. No.: US 2024/0386205 A1
(43) Pub. Date: Nov. 21, 2024

(54) INFORMATION PROCESSING DEVICE, INFORMATION PROCESSING METHOD, AND NON-TRANSITORY STORAGE MEDIUM

(71) Applicant: NEC Corporation, Minato-ku, Tokyo (JP)

(72) Inventor: Yutaka UNO, Tokyo (JP)

(73) Assignee: NEC Corporation, Minato-ku, Tokyo (JP)

(21) Appl. No.: 18/692,425

(22) PCT Filed: Sep. 28, 2021
(86) PCT No.: PCT/JP2021/035610

§ 371 Date: Mar. 15, 2024

(51) Int. Cl. G06F 40/284; G16H 10/60

(52) U.S. Cl. CPC G06F 40/284; G16H 10/60

(57) ABSTRACT

In order to attain an object to provide a technique in which calculation cost and a processing capability are well balanced and which is applicable to natural language processing in medical practice, an information processing apparatus includes: an acquisition means (21) for acquiring a token sequence obtained from a medical sentence in an electronic medical record and a context information vector obtained from context information of the electronic medical record; and an output sequence generation means (22) for carrying out an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

### TECHNICAL FIELD

The present invention relates to an information processing apparatus, an information processing method, and a program that each (i) have a processing capability which enables more accurate understanding of a meaning of a sentence and (ii) achieve language processing in which calculation cost and the processing capability are well balanced.

### BACKGROUND ART

A natural language processing technique in which deep learning is used has recently been put into practical use, and its capability has been greatly improved. As a result, such a natural language processing technique has been promoted to be applied in various fields. For example, natural language processing in which deep learning is used has been promoted to be applied also in a medical sentence.

For example, proposed is a technique in which a similarity index value calculated by analyzing sentences contained in an electronic medical record of a patient is used to predict, from the contents of the sentences contained in the electronic medical record, the possibility that the patient will take a dangerous action (for example, see Patent Literature 1).

Furthermore, proposed is a technique in which a degree of similarity between medical practices described in medical data (e.g., receipt information, electronic medical record information) is calculated from another medical practice and a disease name that are written around and along with the medical practices, and similar medical practices are extracted as a group (for example, see Patent Literature 2).

### CITATION LIST

### Patent Literature

### [Patent Literature 1]

International Patent Application Publication No. WO2019/212005

### [Patent Literature 2]

Japanese Patent Application Publication, Tokukai, No. 2019-212034

### SUMMARY OF INVENTION

### Technical Problem

However, for example, in order to accurately analyze a medical sentence in Patent Literature 1, an extremely high-performance information processing apparatus such as a GPU is necessary. This unfortunately imposes greater burden on medical practice in terms of cost. In addition, in the technique of Patent Literature 2, an order of appearance of words in medical sentences is not considered. Thus, it may be determined that medical sentences having different meanings are similar medical sentences.

An example aspect of the present invention has been made in view of the above problems, and an example object thereof is to provide a technique in which calculation cost and a processing capability are well balanced and which is applicable to natural language processing in medical practice.

### Solution to Problem

An information processing apparatus according to an example aspect of the present invention includes: an acquisition means for acquiring a token sequence obtained from a medical sentence in an electronic medical record and a context information vector obtained from context information of the electronic medical record; and an output sequence generation means for carrying out an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

An information processing method according to an example aspect of the present invention includes: acquiring a token sequence obtained from a medical sentence in an electronic medical record and a context information vector obtained from context information of the electronic medical record; and carrying out an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

A program according to an example aspect of the present invention causes a computer to function as an information processing apparatus including: an acquisition means for acquiring a token sequence obtained from a medical sentence in an electronic medical record and a context information vector obtained from context information of the electronic medical record; and an output sequence generation means for carrying out an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

### Advantageous Effects of Invention

An example aspect of the present invention makes it possible to provide a technique in which calculation cost and a processing capability are well balanced and which is applicable to natural language processing in medical practice.

### BRIEF DESCRIPTION OF DRAWINGS

FIG. 1 is a block diagram illustrating an example configuration of an information processing apparatus according to a first example embodiment of the present invention.

FIG. 2 is a flowchart showing a flow of an information processing method according to the first example embodiment of the present invention.

FIG. 3 is a block diagram illustrating an example configuration of a natural language processing apparatus according to a second example embodiment of the present invention.

FIG. 4 is a diagram obtained by modeling an output sequence generation process carried out by an output sequence generation section.

FIG. 5 is a flowchart describing an example of natural language processing according to the second example embodiment of the present invention.

FIG. 6 is a flowchart describing an example of an output process.

FIG. 7 is a flowchart describing an example of a high-dimensional feature vector transformation process.

FIG. 8 is a diagram describing examples of data input to a natural language processing apparatus and data output as a result of carrying out natural language processing.

FIG. 9 is a diagram describing an input vector space and a high-dimensional feature space.

FIG. 10 is a block diagram illustrating an example configuration of a natural language learning and processing apparatus according to a third example embodiment of the present invention.

FIG. 11 is a flowchart describing an example of a learning process according to the third example embodiment of the present invention.

FIG. 12 is a flowchart describing an example of a parameter updating process.

FIG. 13 is a flowchart describing an example of an output weight matrix learning process.

FIG. 14 is a diagram illustrating examples of medical sentences in which identical words are used.

FIG. 15 is a block diagram illustrating an example configuration of a natural language learning apparatus according to a fourth example embodiment of the present invention.

FIG. 16 is a block diagram illustrating an example hardware configuration of an apparatus according to each of the example embodiments of the present invention.

### EXAMPLE EMBODIMENTS

### First Example Embodiment

The following description will discuss a first example embodiment of the present invention in detail with reference to the drawings. The first example embodiment is an embodiment serving as a basis for example embodiments described later.

### <Overview of Information Processing Apparatus 20>

An information processing apparatus 20 according to the present example embodiment is, schematically speaking, an apparatus that predicts whether a word constituting a given sentence is used with a positive meaning or with a negative meaning in the given sentence.

More specifically, for example, the information processing apparatus 20 includes:
an acquisition means for acquiring a token sequence obtained from a medical sentence in an electronic medical record and a context information vector obtained from context information of the electronic medical record; and
an output sequence generation means for carrying out an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

### <Configuration of Information Processing Apparatus 20>

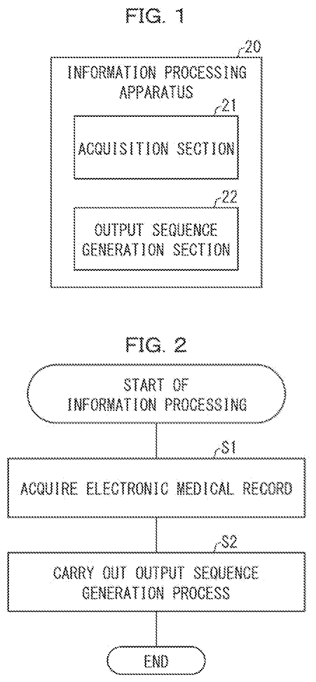

The following description will discuss, with reference to FIG. 1, a configuration of the information processing apparatus 20 according to the present example embodiment. FIG. 1 is a block diagram illustrating an example configuration of the information processing apparatus 20.

As illustrated in FIG. 1, the information processing apparatus 20 includes an acquisition section 21 and an output sequence generation section 22. The acquisition section 21 is configured to realize the acquisition means in the present example embodiment. The output sequence generation section 22 is configured to realize an output sequence generation section means in the present example embodiment.

The acquisition section 21 acquires a token sequence obtained from a medical sentence in an electronic medical record and a context information vector obtained from context information of the electronic medical record.

Note here that the electronic medical record is, for example, data in which information pertaining to medical treatment, including a medical sentence is electronized, structured, and recorded.

The token sequence is a sequence in which a plurality of tokens are arranged. For example, tokens are generated by vectorizing words contained in a medical sentence, and a token is generated by arranging the tokens in sequence correspondence with an order in which the words appear.

The context information is, for example, structured information contained in a structured electronic medical record, and is, for example, information other than text recorded as a medical sentence. The context information includes, for example, information such as a job title of a person who has written a medical sentence and a date and time when the medical sentence was recorded. The context information is also vectorized so as to be a context information vector.

The output sequence generation section 22 carries out an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

The high-dimensional feature vector is generated by, for example, multiplying a token and vectorized context information by a predetermined weight matrix. The output sequence is generated by multiplying the high-dimensional feature vector by a weight matrix obtained by pre-training. In this case, the weight matrix is determined so that the high-dimensional feature vector has a higher dimension than a sum of a dimension of a vector serving as each token in the token sequence and the dimension of the context information vector.

### <Effect of Information Processing Apparatus 20>

According to the information processing apparatus 20 according to the present example embodiment, an output sequence generation process for generating an output sequence from a token sequence and a context information vector, the output sequence generation process including a process for transformation into a high-dimensional feature vector that has a higher dimension than a sum of a dimension of the token sequence and a dimension of the context information vector, is carried out. This makes it possible to more accurately and easily understand a meaning of a medical sentence.

That is, since the output sequence generation process including a process for transformation into a high-dimensional feature vector that has a higher dimension than a sum of a dimension of the token sequence and a dimension of the context information vector is carried out, it is possible to more accurately understand a meaning of a sentence. Furthermore, in the present embodiment, the output sequence is generated from the token sequence and the context information vector. This makes it possible to, for example, more easily understand a meaning of a medical sentence, as compared with a case where the meaning of the medical sentence is understood only from a word of the medical sentence.

### <Flow of Information Processing Method Carried Out by Information Processing Apparatus 20>

The following description will discuss, with reference to FIG. 2, a flow of an information processing method that is carried out by the information processing apparatus 20 configured as described above. FIG. 2 is a flowchart showing the flow of the information processing method. As illustrated in FIG. 2, information processing includes a step S1 and a step S2.

In the step S1, the acquisition section 21 acquires a token sequence obtained from a medical sentence in an electronic medical record and a context information vector obtained from context information of the electronic medical record.

In the step S2, the output sequence generation section 22 carries out an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

### <Effect of Information Processing Method>

According to the information processing method according to the present example embodiment, since the output sequence generation process including a process for transformation into a high-dimensional feature vector that has a higher dimension than a sum of a dimension of the token sequence and a dimension of the context information vector is carried out, it is possible to more accurately understand a meaning of a sentence. Furthermore, in the present embodiment, the output sequence is generated from the token sequence and the context information vector. This makes it possible to, for example, more easily understand a meaning of a medical sentence, as compared with a case where the meaning of the medical sentence is understood only from a word of the medical sentence.

### Second Example Embodiment

The following description will discuss a second example embodiment of the present invention in detail with reference to the drawings. Note that members having functions identical to those of the respective members described in the first example embodiment are given respective identical reference numerals, and a description of those members is omitted as appropriate.

### <Configuration of Natural Language Processing Apparatus 10>

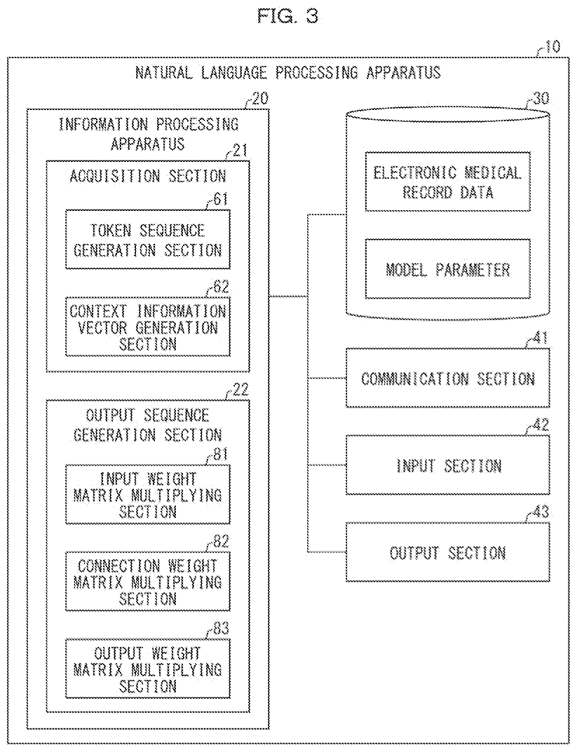

The following description will discuss, with reference to FIG. 3, a configuration of a natural language processing apparatus 10 according to the present example embodiment. FIG. 3 is a block diagram illustrating an example configuration of the natural language processing apparatus 10. As illustrated in FIG. 3, the natural language processing apparatus 10 includes an information processing apparatus 20, a storage section 30, a communication section 41, an input section 42, and an output section 43.

The information processing apparatus 20 is a functional block which has a function similar to that of the information processing apparatus 20 described in the first example embodiment.

The storage section 30 is constituted by, for example, a semiconductor memory device and stores data. In this example, the storage section 30 stores electronic medical record data and a model parameter. Note here that the model parameter is a weighting factor obtained by machine learning (described later).

The communication section 41 is an interface for connecting the natural language processing apparatus 10 to a network. A specific configuration of the network is not limited to the present example embodiment. Examples of the network include a wireless local area network (LAN), a wired LAN, a wide area network (WAN), a public network, a mobile data communication network, and a combination of these networks.

The input section 42 receives various inputs to the natural language processing apparatus 10. A specific configuration of the input section 42 is not limited to the present example embodiment. For example, the input section 42 can be configured to include any of input devices such as a keyboard and a touch pad. The input section 42 may also be configured to include, for example, a data scanner that reads data via electromagnetic waves such as infrared rays and radio waves, and a sensor that senses a state of an environment.

The output section 43 is a functional block that outputs a processing result from the natural language processing apparatus 10. A specific configuration of the output section 43 is not limited to the present example embodiment. For example, the output section 43 is constituted by, for example, a display, a speaker, or a printer, and displays various processing results, etc. from the natural language processing apparatus 10 on a screen, or outputs the various processing results, etc. as a sound or drawing.

In the example of FIG. 3, the acquisition section 21 includes a token sequence generation section 61 and a context information vector generation section 62.

The token sequence generation section 61 generates a token sequence by transforming each word contained in a medical sentence into a token by embedding the each word in a first vector space defined in advance. Note here that the first vector space is configured such that a word which may be contained in the medical sentence can be embedded therein. For example, the first vector space is obtained by using a language processing model to learn, in advance, the word that may be contained in the medical sentence.

The language processing model is not limited to the present example embodiment and may be, for example, Skipgram. Skipgram is a kind of Word2vec and is a language processing model that can predict, in a case where a certain word is input, what word easily appears around the certain word. Dimensionality of a vector in the first vector space is arbitrary. Note, however, that the vector is desirably not-too-high-dimensional in order to carry out, at a high speed, a calculation process described later.

The context information vector generation section 62 generates a context information vector by extracting predetermined context information from an electronic medical record and embedding the predetermined context information in a second vector space defined in advance. As described earlier, context information is, for example, information other than text recorded as a medical sentence and is information indicative of, for example, an attribute of the medical sentence. For example, in a case where the context information is a job title of a person who has written a medical sentence, information such as “doctor”, “nurse”, “emergency medical technician”, “physiotherapist”, “nutritionist”, . . . , or the like is embedded in the second vector space so that the context information vector is generated.

Note that the second vector space may be obtained by pre-training or may be a predetermined vector space. Dimensionality of a vector in the second vector space is arbitrary. Note, however, that the vector is desirably not-too-high-dimensional in order to carry out, at a high speed, the calculation process described later.

In this way, a word and context information can be appropriately vectorized.

Furthermore, in the example of FIG. 3, the output sequence generation section 22 provided in the information processing apparatus 20 includes an input weight matrix multiplying section 81, a connection weight matrix multiplying section 82, and an output weight matrix multiplying section 83. The output sequence generation section uses, for example, an echo state network (ESN), which is one of recurrent neural network (RNN) models, to predict (generate) an output sequence.

The ESN includes an input layer, a reservoir layer, and an output layer, and enables input information to be linearly separable by mapping a low-dimensional vector of the input layer into a high-dimensional neuronal state space by non-linear transformation. In order to learn a correlation between input data and output data, only the output layer needs to learn the correlation, and the input layer and the reservoir layer do not need to learn the correlation. Thus, the ESN further reduces calculation cost in machine learning as compared with a common RNN.

The input weight matrix multiplying section 81 calculates a product of a predetermined input weight matrix and a token, and calculates a product of a predetermined context weight matrix and a context information vector. Note that the input weight matrix and the context weight matrix each can be arbitrarily set as a matrix which does not change dimensionality of the token and of the context information vector. That is, it is not necessary to learn an input weight vector and the context weight matrix.

An input vector is generated by combining vectors of the products thus obtained.

The connection weight matrix multiplying section 82 transforms the input vector into a high-dimensional feature vector by multiplying the input vector by a predetermined connection weight matrix. As described earlier, the high-dimensional feature vector has a higher dimension than a sum of a dimension of the token and a dimension of the context information vector. For example, in a case where a vector and the token as a vector are three-dimensional and the context information vector is one-dimensional, the high-dimensional feature vector is a vector whose number of dimensions is more than 4 (1+3), i.e., 5 or more.

In this way, the token and the context information can be transformed into a high-dimensional vector.

The connection weight matrix can be arbitrarily set as a matrix that transforms a dimension of an input vector into a dimension of a high-dimensional feature vector. That is, it is not necessary to learn the connection weight matrix.

The output weight matrix multiplying section 83 multiplies a high-dimensional feature vector by an output weight matrix obtained by pre-training. With this, the high-dimensional feature vector is transformed into a lower-dimensional output vector, and an output sequence is generated by arranging output vectors in correspondence with an order of token sequences. The output weight matrix is regarded as a matrix that transforms a dimension of the high-dimensional feature vector into a dimension of an output vector. A component of the output weight matrix is found by pre-training and is stored, as the model parameter, in the storage section 30 in FIG. 3.

In this way, the output sequence is generated by multiplying the high-dimensional feature vector by the output weight matrix obtained by pre-training. This enables only the output weight matrix to be a weight matrix that needs learning.

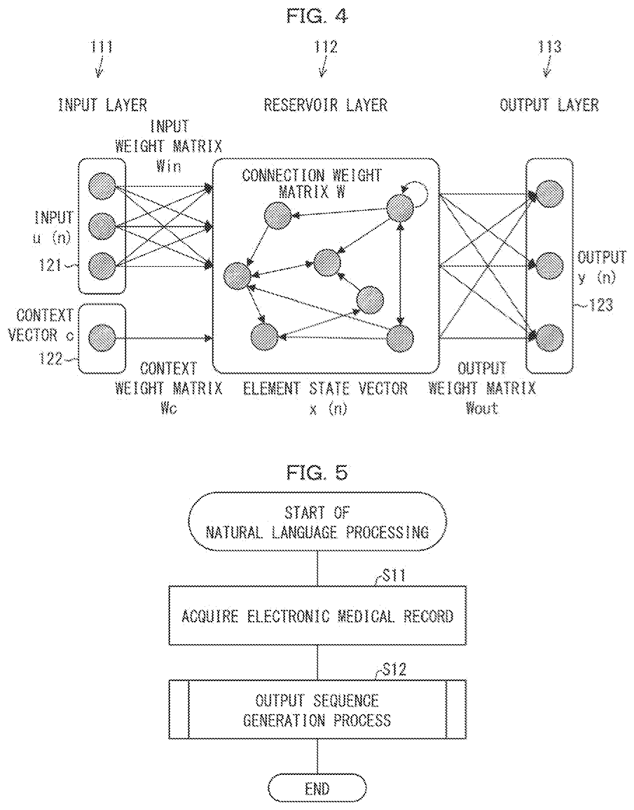

FIG. 4 is a diagram obtained by modeling an output sequence generation process carried out by the output sequence generation section 22. A model illustrated in FIG. 4 uses an ESN and has an input layer 111, a reservoir layer 112, and an output layer 113.

In this example, each token in an input token sequence is assumed to be a three-dimensional vector, and is represented by an input u (n). Note here that “n” is regarded as a value indicative of an order of a token in the token sequence, and indicates, for example, an order in which a word corresponding to the token appears in a medical sentence. In the input layer 111, the input u (n) is multiplied by an input weight matrix Win. The input weight matrix Win is regarded as a matrix that does not change dimensions of a vector and of the token as a vector, and is arbitrarily set.

Furthermore, in this example, the context information vector is assumed to be a one-dimensional vector, and is represented by a context vector c. In the input layer 111, the context vector c is multiplied by a context weight matrix Wc. The context weight matrix Wc is regarded as a matrix that does not change a dimension of the context information vector, and is arbitrarily set.

A vector obtained by combining (i) a three-dimensional vector obtained by multiplying the input u (n) by the input weight matrix Win and (ii) a one-dimensional vector obtained by multiplying the context vector c by the context weight matrix Wc is an input vector. In this example, the input vector is a four-dimensional vector.

In the reservoir layer 112, the input vector is multiplied by a connection weight matrix W and transformed into a high-dimensional feature vector. In this example, the input vector is transformed into the high-dimensional feature vector that is seven-dimensional. The connection weight matrix W is regarded as a matrix that transforms the dimension (four-dimension) of the input vector into the dimension (seven-dimension) of the high-dimensional feature vector, and is arbitrarily set.

In the output layer 113, the high-dimensional feature vector is multiplied by an output weight matrix Wout so that an output vector is generated. Here, the output vector is assumed to be a three-dimensional vector, and is represented by an output y (n). The output weight matrix Wout is regarded as a matrix that transforms the dimension (seven-dimension) of the high-dimensional feature vector into the dimension (three-dimension) of the output vector, and a component of the matrix is set by machine learning which is carried out in advance. Machine learning of the output weight matrix is carried out with reference to, for example, training data including a plurality of sets of (i) a medical sentence and context information and (ii) a positive or negative label regarding a given word contained in the medical sentence. This machine learning will be described later in detail in the following example embodiments.

Assume here that a state vector of an element in the reservoir layer 112 is represented by x (n). In this case, state equations of the reservoir layer 112 are represented by the following equations:

$$\begin{matrix}x ( n + 1 ) = f ( {W}^{in} u ( n ) + Wx ( n ) + {W}^{c} c ) \\ y ( n + 1 ) = {W}^{out} x ( n + 1 )\end{matrix}$$

In this way, the ESN is used to predict the output y (n) from the input u (n) and the context vector c.

### <Flow of Natural Language Processing Carried Out by Natural Language Processing Apparatus 10>

The following description will discuss, with reference to FIGS. 5 to 7, a flow of natural language processing that is carried out by the natural language processing apparatus 10 configured as described above.

Since the natural language processing illustrated in FIG. 5 is similar to the process described earlier with reference to FIG. 2, a specific description thereof is omitted here.

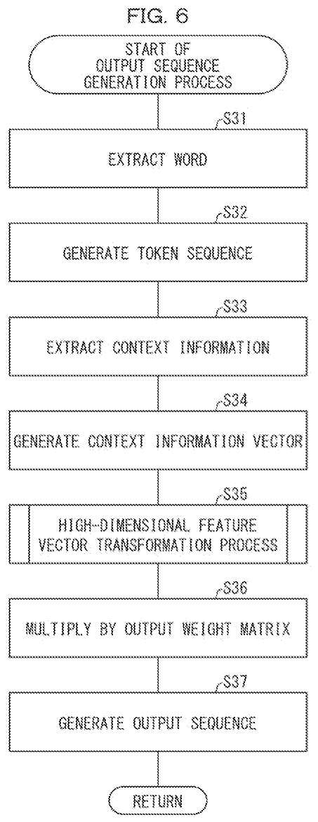

FIG. 6 is a flowchart describing an example of an output sequence generation process that is carried out in a step S12 in FIG. 5. In the example of FIG. 6, the output sequence generation process includes a step S31 to a step S37.

In the step S31, the token sequence generation section 61 extracts a word from a medical sentence in an electronic medical record acquired in a step S11 in FIG. 5. In so doing, by, for example, morphological analysis of a medical sentence recorded as text data, the token sequence generation section 61 extracts a word contained in a medical sentence. It is assumed that for example, n words contained in a medical sentence are each extracted in the step S31.

In the step S32, the token sequence generation section 61 generates a token sequence by transforming each word contained in a medical sentence into a token by embedding the each word in a first vector space defined in advance. As described earlier, the first vector space is obtained by using a language processing model to learn, in advance, a word that may be contained in the medical sentence. The language processing model is not limited to the present example embodiment and may be, for example, Skipgram.

In the step S33, the context information vector generation section 62 extracts predetermined context information from the electronic medical record.

In the step S34, the context information vector generation section 62 generates a context information vector by embedding the context information in a second vector space defined in advance.

Thus, the word and the context information are appropriately vectorized.

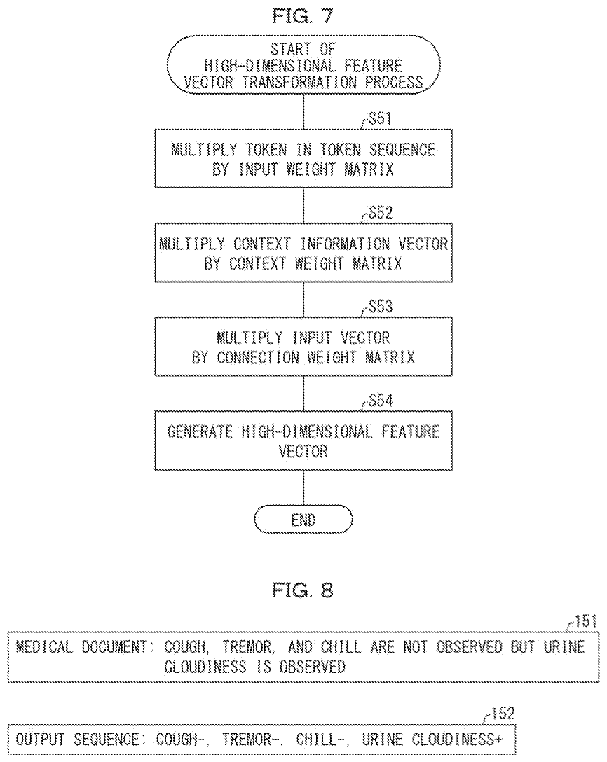

In the step S35, the input weight matrix multiplying section 81 and the connection weight matrix multiplying section 82 carry out a high-dimensional feature vector transformation process. An example of the high-dimensional feature vector transformation process in the step S35 in FIG. 6 is described here with reference to a flowchart of FIG. 7.

In a step S51, the input weight matrix multiplying section 81 multiplies a token in a token sequence by a predetermined input weight matrix. This process corresponds to a process in which the input u (n) is multiplied by the input weight matrix Win in the input layer 111 illustrated in FIG. 4.

In a step S52, the input weight matrix multiplying section 81 multiplies a predetermined context information vector by a context weight matrix. This process corresponds to a process in which the context vector c is multiplied by the context weight matrix Wc in the input layer 111 illustrated in FIG. 4.

In a step S53, the input weight matrix multiplying section 81 multiplies an input vector by a predetermined connection weight matrix, the input vector including a product of an input weight matrix and a token and a product of a context weight matrix and a context information vector. This process corresponds to a process in which the input vector is multiplied by the connection weight matrix W in the reservoir layer 112 illustrated in FIG. 4.

In a step S54, the input weight matrix multiplying section 81 generates a high-dimensional feature vector.

In this way, the high-dimensional feature vector transformation process is carried out. This results in transformation of a token and context information into a high-dimensional vector.

With FIG. 6 referred to again, in the step S36, the output weight matrix multiplying section 83 multiplies the high-dimensional feature vector obtained as a result of the process in the step S35 by an output weight matrix obtained by pre-training. Note that the output weight matrix multiplying section 83 reads out a model parameter stored in the storage section 30, and uses the model parameter as a component of the output weight matrix. This process corresponds to a process in which the high-dimensional feature vector is multiplied by the output weight matrix Wout in the output layer 113 illustrated in FIG. 4. This results in generation of an output vector y (n).

In this way, an output sequence is generated by multiplying the high-dimensional feature vector by the output weight matrix obtained by pre-training. This enables only the output weight matrix to be a weight matrix that needs learning. In the step S37, the output weight matrix multiplying section 83 generates the output sequence. In so doing, the output weight matrix multiplying section 83 generates the output sequence by arranging output vectors in correspondence with an order of token sequences.

Note that the output sequence does not necessarily contain an output vector corresponding to all tokens contained in a token sequence. For example, only an output vector corresponding to a noun among words in a sentence may be contained in the output sequence.

In this way, the output sequence generation process is carried out.

FIG. 8 is a diagram describing examples of data input to the natural language processing apparatus 10 and data output as a result of carrying out natural language processing by the natural language processing apparatus 10. In this example, text data 151 “a cough, a tremor, and a chill are not observed but urine cloudiness is observed” that is a medical document in an electronic medical record is input to the natural language processing apparatus 10. Output data is, for example, an output sequence 152 that is data “cough−, tremor−, chill−, urine cloudiness+”.

In this output sequence, “−” represents a negative label, and “+” represents a positive label. That is, this shows that in an input medical sentence, words “cough”, “tremor”, and “chill” are each used with a negative meaning, and a word “urine cloudiness” is used with a positive meaning. In other words, in the case of this example, it is shown that for a patient's symptom or condition corresponding to the electronic medical record, there is no “cough”, “tremor”, or “chill”, and there is “urine cloudiness”.

Note that each output vector contained in the output sequence is a vector representing a combination of a word and a label.

As described earlier with reference to FIG. 4, in the present embodiment, the input vector of the input layer is transformed into the high-dimensional feature vector in the reservoir layer 112. This allows features of words whose meanings are difficult in the input layer 111 to distinguish to be easily classified by some criterion in the reservoir layer 112.

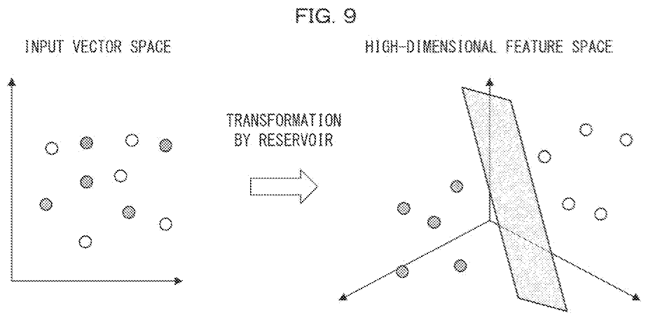

For example, an input vector space (e.g., the input layer 111 in FIG. 4), which is a low-dimensional space, and a high-dimensional feature space (e.g., the reservoir layer 112 in FIG. 4) are considered as illustrated in FIG. 9. The input vector space and the high-dimensional feature space that are illustrated in the example of FIG. 9 are a two-dimensional space and a three-dimensional space, respectively. Black circles and white circles in FIG. 9 represent words in a sentence. In the input vector space, the words are scattered in the two-dimensional space, and it is difficult to distinguish features of the words.

Transformation of the input vector space into the high-dimensional feature space in the reservoir layer makes it easier to classify the features of the words by some criterion. In the example of FIG. 8, features of the words represented by the black circles and the words represented by the white circles are classified by a plane in the high-dimensional feature space in the three-dimensional space.

Furthermore, by, for example, adding, averaging, or combining output vectors generated in the process in the step S36, the output weight matrix multiplying section 83 may further generate a sentence vector corresponding to the input medical sentence. For example, comparison between sentence vectors of two medical sentences by the information processing apparatus 20 makes it possible to determine a degree of semantic similarity between those sentences.

### <Effect of Natural Language Processing Apparatus and Natural Language Processing>

As described above, an RNN language processing model including an input layer, a reservoir layer, and an output layer is employed in the natural language processing apparatus and the natural language processing according to the present example embodiment. Thus, for example, as compared with a language processing model such as Word2vec, the RNN language processing model makes it possible to greatly reduce calculation cost during learning while making it possible to more accurately analyze a meaning of a sentence.

Furthermore, in the natural language processing apparatus and the natural language processing according to the present example embodiment, a configuration is employed such that an input vector composed of a token and a context information vector is transformed into a high-dimensional feature vector. Thus, as compared with a case where only a word of a medical document is merely input, the configuration makes it possible to more accurately predict a label of the word.

### Third Example Embodiment

Next, the following description will discuss a third example embodiment of the present invention in detail with reference to the drawings. Note that members having functions identical to those of the respective members described in the first and second example embodiments are given respective identical reference numerals, and a description of those members is omitted as appropriate.

### <Configuration of Natural Language Learning and Processing Apparatus 10A>

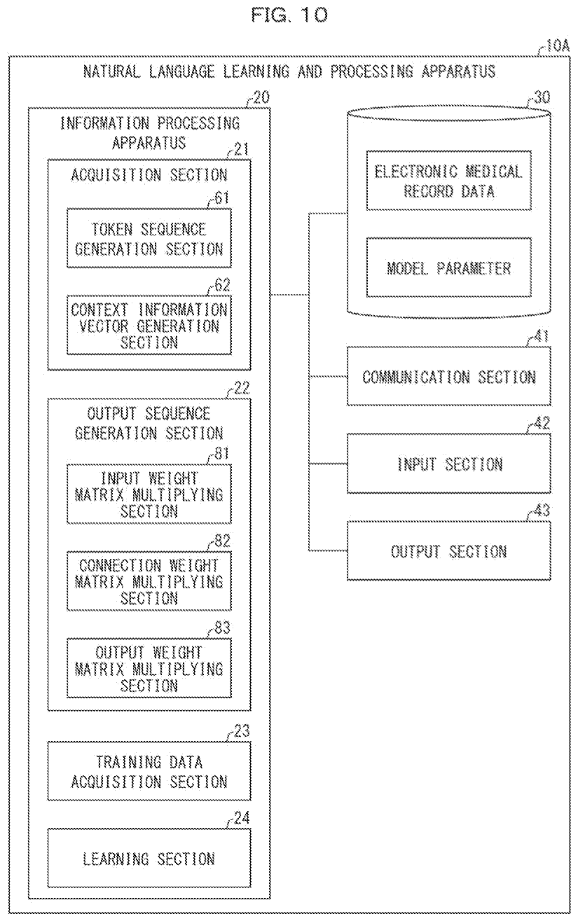

The following description will discuss, with reference to FIG. 10, a configuration of a natural language learning and processing apparatus 10A according to the present example embodiment. The natural language learning and processing apparatus 10A is an apparatus that further has a function of learning the model parameter (the output weight matrix Wout described earlier with reference to FIG. 4) of the storage section 30 provided in the natural language processing apparatus 10.

FIG. 10 is a block diagram illustrating an example configuration of the natural language learning and processing apparatus 10A. The natural language learning and processing apparatus 10A illustrated in FIG. 10 differs from the natural language processing apparatus 10 illustrated in FIG. 3 in that an information processing apparatus 20 includes a training data acquisition section 23 an a learning section 24.

The training data acquisition section 23 acquires training data including a plurality of sets of (i) a medical sentence and context information and (ii) a positive or negative label regarding a given word contained in the medical sentence. The learning section 24 trains an output weight matrix with reference to the training data acquired by the training data acquisition section 23. This enables each component of the output weight matrix to be trained as a parameter of a prediction model.

As described earlier, the training data includes a medical sentence. The medical sentence is a sentence recorded as text data in an electronic medical record, and examples of the medical sentence include the text data 151 illustrated in FIG. 8.

Furthermore, as described earlier, the training data includes context information. The context information is structured information contained in a structured electronic medical record and is information other than text data. Examples of the context information include information indicative of a job title of a person who has written a medical sentence.

Moreover, as described earlier, the training data includes a positive or negative label regarding a given word contained in a medical sentence. The given word means, for example, a noun of a word in a sentence. The positive or negative label indicates whether a given word is used in a medical sentence with a negative meaning or with a positive meaning.

Examples of the given word and the positive or negative label include the words “cough”, “tremor”, “chill”, and “urine cloudiness” in the output sequence 152 illustrated in FIG. 8, and the “+” or “−” labels assigned to those words.

The training data is generated, for example, as below. A medical sentence recorded as text data in an electronic medical record of a patient is acquired by the training data acquisition section 23, and a word (e.g., a noun) to be labeled is extracted from the text data. The word is extracted by, for example, carrying out morphological analysis of the text data by the training data acquisition section 23. Context information corresponding to the text data is also acquired by the training data acquisition section 23.

The acquired sentence of the text data and the extracted word are each displayed on a display of an output section 43. For example, an operator of the natural language learning and processing apparatus 10A reads a medical sentence and determines whether each of extracted words is used with a negative meaning or with a positive meaning. Then, the operator of the natural language learning and processing apparatus 10A assigns, via an input section 42, a positive label (e.g., “+”) or a negative label (e.g., “−”) to each of the words.

Thereafter, a medical sentence recorded as text data in an electronic medical record of another patient is acquired by the training data acquisition section 23, and a similar operation is carried out. By repeatedly carrying out such an operation, training data is generated, the training data including a plurality of sets of (i) a medical sentence and context information and (ii) a positive or negative label regarding a given word contained in the medical sentence.

Note that an operation described earlier for generation of training data is an example and is not limited to the present example embodiment. Note also that the expression “training data” in the present specification is not intended to be limited to data other than data which is referred to for updating (learning) a model parameter. Instead of the expression “training data” in the present specification, an expression such as “learning data” or “reference data” may be alternatively used.

After training data having a sufficient number of sets is generated, machine learning by the learning section 24 is carried out. That is, the learning section 24 refers to the training data and learns a prediction model that represents a correlation between (a) a medical sentence and context information and (b) a positive or negative label regarding a given word contained in the medical sentence.

In this case, machine learning is carried out by updating a model parameter of the prediction model so that a difference between a positive or negative label regarding a given word, the positive or negative label being output by the prediction model, and a positive or negative label regarding a given word contained in training data is further reduced. Note here that the prediction model is a model in which the ESN described earlier with reference to FIG. 4 is used and that the model parameter is the output weight matrix Wout described earlier.

### <Flow of Learning Process Carried Out by Natural Language Learning and Processing Apparatus 10A>

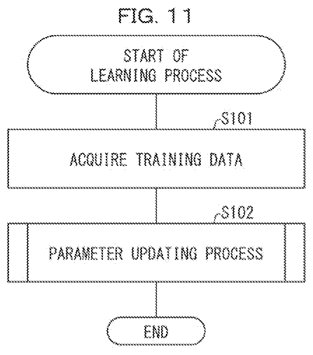

The following description will discuss, with reference to FIG. 11, a flow of a learning process that is carried out by the natural language learning and processing apparatus 10A configured as described above. FIG. 11 is a flowchart showing the flow of the learning process.

In a step S101, the training data acquisition section 23 acquires training data including a plurality of sets of (i) a medical sentence and context information and (ii) a positive or negative label regarding a given word contained in the medical sentence.

In a step S102, the learning section 24 refers to the training data acquired in the step S101, and carries out a parameter updating process for training an output weight matrix. Note that the parameter updating process in the step S102 is carried out a plurality of times in accordance with the number of sets included in the training data acquired in the step S101.

This allows the each component of the output weight matrix to be trained as a parameter of a prediction model.

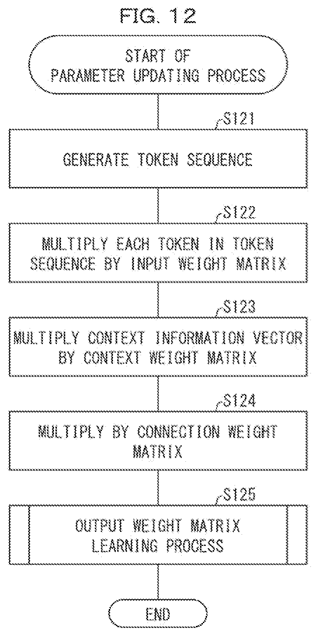

FIG. 12 is a flowchart describing a specific example of the parameter updating process in the step S102 in FIG. 11.

In a step S121, the learning section 24 generates a token sequence from text data included in the acquired training data.

In a step S122, the learning section 24 multiplies each token in the token sequence by an input weight matrix. In this case, the input u (n) is multiplied by the input weight matrix Win as described earlier with reference to FIG. 4.

In a step S123, the learning section 24 multiplies a context information vector by a context weight matrix. In this case, the context vector c is multiplied by the context weight matrix Wc as described earlier with reference to FIG. 4.

In a step S124, the learning section 24 multiplies an input vector by a connection weight matrix, the input vector including a product of an input weight matrix and a token and a product of a context weight matrix and a context information vector. In this case, the input vector is multiplied by the connection weight matrix W as described earlier with reference to FIG. 4. Note that each input vector corresponding to the each token in the token sequence is multiplied by the connection weight matrix W.

In a step S125, the learning section 24 carries out an output weight matrix learning process. This allows the each component of the output weight matrix serving as a model parameter to be calculated and updated with reference to the training data.

In this way, the parameter updating process is carried out.

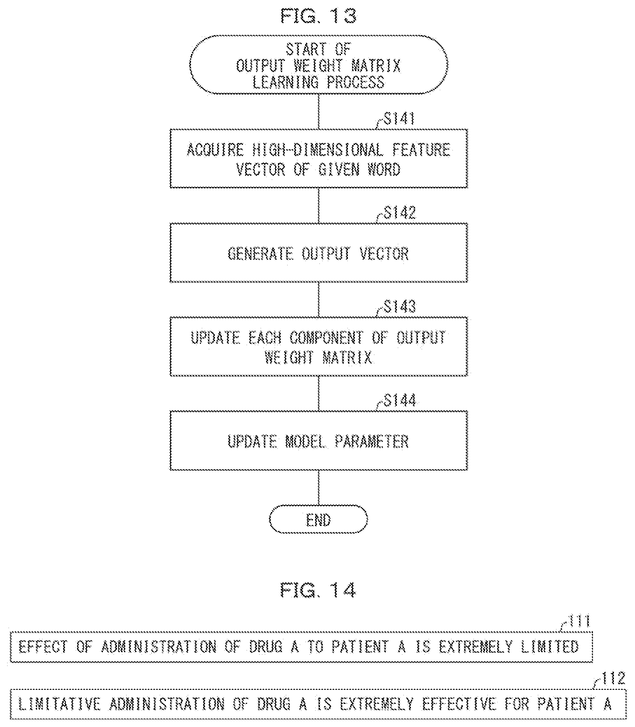

FIG. 13 is a flowchart describing a specific example of the output weight matrix learning process in the step S125 in FIG. 12.

In a step S141, the learning section 24 acquires a high-dimensional feature vector of a given word. Note here that the high-dimensional feature vector is each high-dimensional feature vector generated in the process in the step S124 by multiplying the each input vector by the connection weight matrix. In this case, a high-dimensional feature vector corresponding to a token generated from a given word which is contained in the training data and to which a label is assigned is acquired.

In a step S142, the learning section 24 generates an output vector. In so doing, the learning section 24 acquires a positive or negative label assigned to a given word contained in the training data and generates an output vector obtained by vectorizing such a word and such a label.

In a step S143, the learning section 24 updates the each component of the output weight matrix so that a difference between the high-dimensional feature vector acquired in the process in the step S141 and the output vector generated in the process in the step S142 is reduced.

In a step S144, the learning section 142 updates the model parameter. In this case, the model parameter of the storage section 30 is updated by the each component of the output weight matrix, the each component having been calculated in the process in the step S143.

Note that the processes in the steps S141 to S144 are each carried out in accordance with the number of given words.

As described earlier with reference to FIG. 11, the parameter updating process in the step S102 is carried out a plurality of times in accordance with the number of sets included in the training data acquired in the step S101. Thus, the each component of the output weight matrix is also repeatedly updated a plurality of times. As described above, learning of the output weight matrix advances as the each component of the output weight matrix is updated.

In this way, the output weight matrix learning process is carried out, and the learning process illustrated in FIG. 11 ends.

Upon completion of the learning process, each component stored as the model parameter of the storage section 30 can be used as the output weight matrix Wout described earlier with reference to FIG. 4. That is, in the output sequence generation process in FIG. 6, the stored model parameter is read out by the output weight matrix multiplying section 83 of the output sequence generation section 22 and multiplied by the high-dimensional feature vector.

This makes it possible to learn whether a given word contained in a medical sentence is used with a negative meaning or with a positive meaning. Thus, for example, it is possible to predict the output sequence 152 from the text data 151 illustrated in FIG. 8.

In the learning process described earlier, learning may be carried out by adjusting a hyperparameter as appropriate. For example, a hyperparameter(s) such as a spectral radius, a leak rate, an input scaling, a reservoir size, and/or a transient response period may be adjusted by the learning section 24.

### <Effect of Natural Language Learning and Processing Apparatus and Learning Process>

In this way, in the natural language learning and processing apparatus and the learning process according to the present example embodiment, a correlation between (a) a medical sentence and context information and (b) a (positive or negative) meaning of a given word in the medical sentence can be trained.

That is, since not only a word but also a medical sentence and context information are included training data, it is possible to carry out machine learning also in consideration of a relationship between an order in which a given word appears in a sentence and context information.

In addition, in this machine learning, a model parameter of a prediction model in which the ESN described earlier with reference to FIG. 4 is used is trained.

As described earlier, in the ESN, only an output weight matrix is trained, and an input weight matrix and a connection weight matrix are each regarded as a predetermined matrix and need not be trained. Thus, the ESN makes it possible to greatly minimize calculation cost during learning as compared with a common RNN. This enables a prediction with use of a prediction model without using, for example, an extremely high-performance information processing apparatus.

Furthermore, use of a trained model parameter makes it possible to predict a (positive or negative) meaning of each word in a medical sentence. This enables the meaning of the each word to be reflected also in a sentence vector generated by adding, averaging, or combining output vectors of an output sequence.

For example, in a conventional language processing model such as Word2vec, an order of appearance of a word in a sentence is not considered. Thus, sentence vectors of sentences in which identical words are used closely resemble each other in many cases. However, even the identical words may be used with a positive meaning or with a negative meaning depending on the sentences.

FIG. 14 illustrates examples of medical sentences in which identical words are used. Note here that a medical sentence 111 and a medical sentence 112 contain identical words (nouns) which are “patient A”, “drug A”, and “administration” and that an adjectival verb “limitative” and an adjectival verb “limited” closely resemble each other. However, in an aspect of administration of the drug A to the patient A, the medical sentence 111 and the medical sentence 112 have almost exactly opposite meanings.

According to the first to third example embodiments described in the present specification, for example, an output sequence can be generated so that a degree of similarity between a sentence vector of the medical sentence 111 and a sentence vector of the medical sentence 112 is reduced.

### Fourth Example Embodiment

Next, the following description will discuss a third example embodiment of the present invention in detail with reference to the drawings. Note that members having functions identical to those of the respective members described in the first to third example embodiments are given respective identical reference numerals, and a description of those members is omitted as appropriate.

### <Configuration of Natural Language Learning Apparatus 10B>

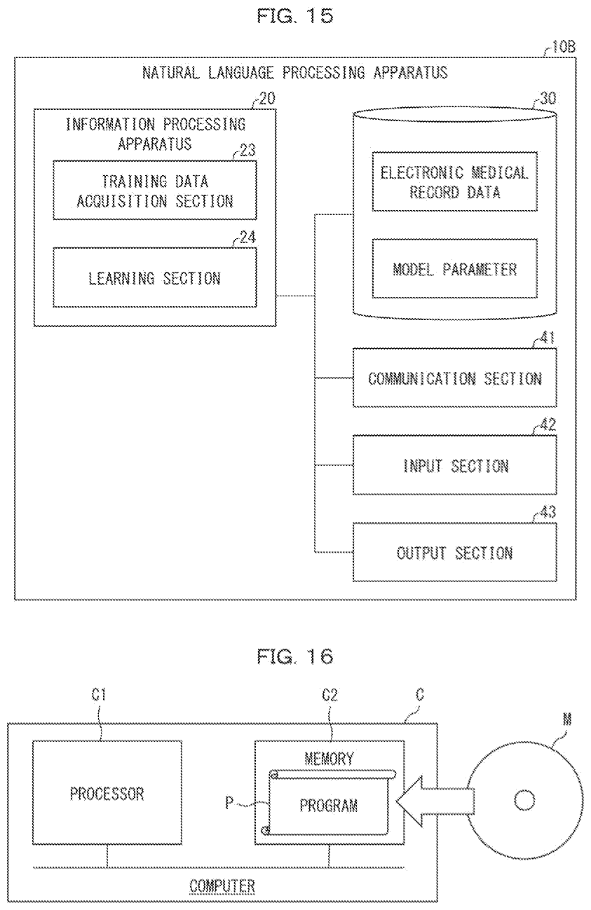

The following description will discuss, with reference to FIG. 15, a configuration of a natural language learning apparatus 10B according to the present example embodiment.

Unlike the natural language learning and processing apparatus 10A described earlier with reference to FIG. 10, the natural language learning apparatus 10B is an apparatus that is configured not to have a function related to generation of an output sequence but to have a function related to learning of a model parameter. That is, in the natural language learning apparatus 10B, neither an acquisition section 21 nor an output sequence generation section 22 is provided in an information processing apparatus 20. Other configurations of the natural language learning apparatus 10B are similar to those of the natural language learning and processing apparatus 10A, and a specific description thereof is omitted here.

In the natural language learning apparatus 10B, the learning process described with reference to FIGS. 11 to 13 is carried out, and learning of a model parameter in a storage section 30 is carried out. After learning with reference to training data having a sufficient number of sets has been carried out, the model parameter stored in the storage section 30 of the natural language learning apparatus 10B is stored in, for example, a storage medium such as a USB memory. Then, the model parameter stored in the storage medium is used by another apparatus (for example, the natural language processing apparatus 10 of FIG. 3). Alternatively, a model parameter of the natural language learning apparatus 10B may be transferred to another apparatus via a network.

### <Effect of Natural Language Learning Apparatus>

As described above, in the natural language learning apparatus according to the present example embodiment, a model parameter for predicting, from a medical sentence and context information, a (positive or negative) meaning of a given word in the medical sentence can be trained.

This makes it possible to, for example, provide model parameters obtained by learning with reference to different training data. Alternatively, model parameters trained by changing adjustment of a hyperparameter may be provided. Examples of the hyperparameter include an ESN spectral radius, an ESN leak rate, an ESN input scaling, an ESN reservoir size, and an ESN transient response period. Use of different model parameters makes it possible to, for example, carry out an optimum prediction in accordance with a characteristic of a patient and/or a type of disease of the patient.

### Fifth Example Embodiment

The present example embodiment will further describe an example of information used as context information.

For example, the context information may be information indicative of a job title of a person who has written a medical sentence. For example, in a case where a job title of a recorder of an electronic medical record is recorded in the electronic medical record, the recorded job title can be set as the information indicative of the job title of the person who has written the medical sentence.

Alternatively, a code indicative of a job title such as “doctor”, “nurse”, “emergency medical technician”, “physiotherapist”, “nutritionist”, . . . , or the like may be set as the information indicative of the job title of the person who has written the medical sentence.

For example, in many cases, emergency medical technicians, physiotherapists, nutritionists, etc. use medical apparatuses different from those used by doctors and/or nurses, and/or describe a patient's condition with attention paid to points different from those to which the doctors and/or the nurses pay attention. Furthermore, in accordance with a job title that is a doctor, a nurse, an emergency medical technician, a physiotherapist, a nutritionist, or the like, there is also, for example, a fixed phrase used in a medical sentence.

Thus, depending on a job title of a person who has written a medical sentence, even sentences in which identical words are used may have different meanings. In a case where a medical sentence is trained in accordance with a job title of a person who has described the medical sentence, even a relatively small amount of learning enables an accurate prediction.

Alternatively, for example, the context information may be a name of a field in which a medical sentence is recorded in an electronic medical record. Assume, for example, that in an electronic medical record, there are “patient's chief complaint”, “examination result”, “follow-up”, . . . , etc. as names of fields in which text should be input. In this case, the names of those fields may be used as the context information. Alternatively, a code indicative of a name of a field may be set as a name of a field in which a medical sentence is recorded.

For example, details described in “patient's chief complaint” is a subjective symptom of a patient himself/herself, whereas details described in “examination result” is an objective symptom based on an examination result. In addition, even in a case where “serious illness” that is understood by the patient is written as a chief complaint, “serious illness” used in the examination result means a state in which an artificial heart and lung and/or a respirator is/are necessary, and is often greatly different from patient's understanding.

Thus, depending on a field in which a medical sentence is recorded, even sentences in which identical words are used may have different meanings. In a case where a medical sentence is trained in accordance with a field in which the medical sentence is recorded, even a relatively small amount of learning enables an accurate prediction.

An example of the context information is described here as a name of a field in which a medical sentence is recorded. Note, however, that the context information may be a name of, for example, a tab, a record, or a table in which a medical sentence is recorded in accordance with a structure of an electronic medical record.

In the example embodiments shown in the present specification, context information is used to predict an output sequence and/or learn an output vector. Thus, a relatively small amount of learning makes it possible to carry out an accurate prediction.

### [Software Implementation Example]

Some or all of functions of the information processing apparatus 20, the natural language processing apparatus 10, the natural language learning and processing apparatus 10A, and the natural language learning apparatus 10B may be realized by hardware such as an integrated circuit (IC chip) or may be alternatively realized by software.

In the latter case, the information processing apparatus 20, the natural language processing apparatus 10, the natural language learning and processing apparatus 10A, and the natural language learning apparatus 10B are each realized by, for example, a computer that executes instructions of a program that is software realizing the functions. FIG. 16 illustrates an example of such a computer (hereinafter referred to as “computer C”).

The computer C includes at least one processor C1 and at least one memory C2. In the memory C2, a program P for causing the computer C to operate as each of the information processing apparatus 20, the natural language processing apparatus 10, the natural language learning and processing apparatus 10A, and the natural language learning apparatus 10B is recorded. In the computer C, the functions of each of the information processing apparatus 20, the natural language processing apparatus 10, the natural language learning and processing apparatus 10A, and the natural language learning apparatus 10B are realized by the processor C1 reading the program P from the memory C2 and executing the program P.

The processor C1 may be, for example, a central processing unit (CPU), a graphic processing unit (GPU), a digital signal processor (DSP), a micro processing unit (MPU), a floating point number processing unit (FPU), a physics processing unit (PPU), a microcontroller, or a combination thereof. The memory C2 may be, for example, a flash memory, a hard disk drive (HDD), a solid state drive (SSD), or a combination thereof.

Note that the computer C may further include a random access memory (RAM) in which the program P is loaded when executed and/or in which various kinds of data are temporarily stored. The computer C may further include a communication interface for transmitting and receiving data to and from another apparatus. The computer C may further include an input/output interface for connecting the computer C to an input/output apparatus(es) such as a keyboard, a mouse, a display, and/or a printer.

The program P can also be recorded in a non-transitory tangible storage medium M from which the computer C can read the program P. Such a storage medium M may be, for example, a tape, a disk, card, a semiconductor memory, a programmable logic circuit, or the like. The computer C can acquire the program P via the storage medium M. The program P can also be transmitted via a transmission medium. Such a transmission medium may be, for example, a communication network, a broadcast wave, or the like. The computer C can acquire the program P also via the transmission medium.

### Additional Remark 1

The present invention is not limited to the foregoing example embodiments, but may be altered in various ways by a skilled person within the scope of the claims. For example, the present invention also encompasses, in its technical scope, any example embodiment derived by appropriately combining technical means disclosed in the foregoing example embodiments.

### [Additional Remark 2]

The whole or part of the example embodiments disclosed above can also be described as below. Note, however, that the present invention is not limited to the following supplementary notes.

### (Supplementary Note 1)

An information processing apparatus including:
an acquisition means for acquiring a token sequence obtained from a medical sentence in an electronic medical record and a context information vector obtained from context information of the electronic medical record; and
an output sequence generation means for carrying out an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

### (Supplementary Note 2)

The information processing apparatus according to Supplementary note 1, wherein
the acquisition means includes
   a token sequence generation means for generating the token sequence by transforming each word contained in the medical sentence into a token by embedding the each word in a first vector space defined in advance, and
   a context information vector generation means for generating the context information vector by extracting predetermined context information from the electronic medical record and embedding the predetermined context information in a second vector space defined in advance.

### (Supplementary Note 3)

The information processing apparatus according to Supplementary note 2, wherein
the output sequence generation means transforms an input vector into the high-dimensional feature vector by multiplying the input vector by a predetermined connection weight matrix, the input vector including a product of a predetermined input weight matrix and the token and a product of a predetermined context weight matrix and the context information vector.

### (Supplementary Note 4)

The information processing apparatus according to Supplementary note 3, wherein
the output sequence generation means further generates the output sequence by multiplying the high-dimensional feature vector by an output weight matrix obtained by pre-training.

### (Supplementary Note 5)

The information processing apparatus according to Supplementary note 4, further including
a learning section that trains the output weight matrix with reference to training data including a plurality of sets of (i) the medical sentence and the context information and (ii) a positive or negative label regarding a given word contained in the medical sentence.

### (Supplementary Note 6)

The information processing apparatus according to any one of Supplementary notes 1 to 5, wherein
the context information is information indicative of a job title of a person who has written the medical sentence.

### (Supplementary Note 7)

The information processing apparatus according to any one of Supplementary notes 1 to 5, wherein
the context information is a name of a field in which the medical sentence is recorded in the electronic medical record.

### (Supplementary Note 8)

An information processing method including:
acquiring a token sequence obtained from a medical sentence in an electronic medical record and a context information vector obtained from context information of the electronic medical record; and
carrying out an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

### (Supplementary Note 9)

The information processing method according to Supplementary note 8, wherein
the output sequence generation process includes
   generating the token sequence by transforming each word contained in the medical sentence into a token by embedding the each word in a first vector space defined in advance, and
   generating the context information vector by extracting predetermined context information from the electronic medical record and embedding the predetermined context information in a second vector space defined in advance.

### (Supplementary Note 10)

The information processing method according to Supplementary note 9, wherein
in the output sequence process,
   an input vector is further transformed into the high-dimensional feature vector by multiplying the input vector by a predetermined connection weight matrix, the input vector including a product of a predetermined input weight matrix and the token and a product of a predetermined context weight matrix and the context information vector.

### (Supplementary Note 11)

The information processing method according to Supplementary note 8, wherein
in the output sequence process,
the output sequence is further generated by multiplying the high-dimensional feature vector by an output weight matrix obtained by pre-training.

### (Supplementary Note 12)

The information processing method according to Supplementary note 11, further including
training the output weight matrix with reference to training data including a plurality of sets of (i) a given word contained in each of the medical sentence and the context information and (ii) a positive or negative label regarding the given word contained in the medical sentence.

### (Supplementary Note 13)

The information processing method according to any one of Supplementary notes 8 to 12, wherein
the context information is information indicative of a job title of a person who has written the medical sentence.

### (Supplementary Note 14)

The information processing method according to any one of Supplementary notes 8 to 12, wherein
the context information is a name of a field in which the medical sentence is recorded in the electronic medical record.

### (Supplementary Note 15)

A program for causing a computer to function as an information processing apparatus including:
an acquisition means for acquiring a token sequence obtained from a medical sentence in an electronic medical record and a context information vector obtained from context information of the electronic medical record; and
an output sequence generation means for carrying out an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

### (Supplementary Note 16)

The program according to Supplementary note 15, wherein
the acquisition means includes
   a token sequence generation means for generating the token sequence by transforming each word contained in the medical sentence into a token by embedding the each word in a first vector space defined in advance, and
   a context information vector generation means for generating the context information vector by extracting predetermined context information from the electronic medical record and embedding the predetermined context information in a second vector space defined in advance.

### (Supplementary Note 17)

The program according to Supplementary note 16, wherein
the output sequence generation means transforms an input vector into the high-dimensional feature vector by multiplying the input vector by a predetermined connection weight matrix, the input vector including a product of a predetermined input weight matrix and the token and a product of a predetermined context weight matrix and the context information vector.

### (Supplementary Note 18)

The information processing apparatus according to Supplementary note 17, wherein
the output sequence generation means further generates the output sequence by multiplying the high-dimensional feature vector by an output weight matrix obtained by pre-training.

### (Supplementary Note 19)

The information processing apparatus according to Supplementary note 18, further including
a learning section that trains the output weight matrix with reference to training data including a plurality of sets of (i) the medical sentence and the context information and (ii) a positive or negative label regarding a given word contained in the medical sentence.

### (Supplementary Note 20)

The information processing apparatus according to any one of Supplementary notes 15 to 19, wherein
the context information is information indicative of a job title of a person who has written the medical sentence.

### (Supplementary Note 21)

The information processing apparatus according to any one of Supplementary notes 15 to 19, wherein
the context information is a name of a field in which the medical sentence is recorded in the electronic medical record.

### [Additional Remark 3]

The whole or part of the fourth example embodiment can also be described as below. Note, however, that the present invention is not limited to the following supplementary notes.

### (Supplementary Note 22)

A learning apparatus including a learning means for,
with reference to training data including a plurality of sets of (i) a medical sentence in an electronic medical record and context information and (ii) a positive or negative label regarding a given word contained in the medical sentence,
learning an output weight matrix indicative of a correlation between a high-dimensional feature vector and an output sequence, the high-dimensional feature vector being obtained by transformation of a token sequence obtained from the medical sentence and a context information vector obtained from the context information, and having a higher dimension than a sum of a dimension of the token sequence and a dimension of the context information vector, the output sequence being composed of an output vector obtained by vectorizing the given word contained in the medical sentence and the positive or negative label regarding the given word.

### (Supplementary Note 23)

A learning method including
with reference to training data including a plurality of sets of (i) a medical sentence in an electronic medical record and context information and (ii) a positive or negative label regarding a given word contained in the medical sentence,
learning an output weight matrix indicative of a correlation between a high-dimensional feature vector and an output sequence, the high-dimensional feature vector being obtained by transformation of a token sequence obtained from the medical sentence and a context information vector obtained from the context information, and having a higher dimension than a sum of a dimension of the token sequence and a dimension of the context information vector, the output sequence being composed of an output vector obtained by vectorizing the given word contained in the medical sentence and the positive or negative label regarding the given word.

### (Supplementary Note 24)

A program for causing a computer to function as a learning apparatus including a learning means for,
with reference to training data including a plurality of sets of (i) a medical sentence in an electronic medical record and context information and (ii) a positive or negative label regarding a given word contained in the medical sentence,
learning an output weight matrix indicative of a correlation between a high-dimensional feature vector and an output sequence, the high-dimensional feature vector being obtained by transformation of a token sequence obtained from the medical sentence and a context information vector obtained from the context information, and having a higher dimension than a sum of a dimension of the token sequence and a dimension of the context information vector, the output sequence being composed of an output vector obtained by vectorizing the given word contained in the medical sentence and the positive or negative label regarding the given word.

### [Additional Remark 3]

The whole or part of the example embodiments disclosed above can also be expressed as follows.

An information processing apparatus including at least one processor, the at least one processor carrying out:
an acquisition process for acquiring a token sequence obtained from a medical sentence in an electronic medical record and a context information vector obtained from context information of the electronic medical record; and
an output sequence generation process for carrying out an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

Note that the information processing apparatus may further include a memory, which may store a program for causing the at least one processor to carry out the acquisition process and the output sequence generation process. Alternatively, the program may be stored in a non-transitory tangible computer-readable storage medium.

### REFERENCE SIGNS LIST

10 Natural language processing apparatus
10A Natural language learning and processing apparatus
10B Natural language learning apparatus
20 Information processing apparatus
21 Acquisition section
22 Output sequence generation section
23 Training data acquisition section
24 Learning section
30 Storage section
41 Communication section
42 Input section
43 Output section
61 Token sequence generation section
62 Context information vector generation section
81 Input weight matrix multiplying section
82 Connection weight matrix multiplying section
83 Output weight matrix multiplying section

What is claimed is:

1. An information processing apparatus comprising at least one processor, the at least one processor carrying out:
an acquisition process for acquiring a token sequence obtained from a medical sentence in an electronic medical record and a context information vector obtained from context information of the electronic medical record; and
an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

2. The information processing apparatus according to claim 1, wherein
the acquisition process includes
a token sequence generation process for generating the token sequence by transforming each word contained in the medical sentence into a token by embedding the each word in a first vector space defined in advance, and
a context information vector generation process for generating the context information vector by extracting predetermined context information from the electronic medical record and embedding the predetermined context information in a second vector space defined in advance.

3. The information processing apparatus according to claim 2, wherein
in the output sequence generation process, an input vector is transformed into the high-dimensional feature vector by multiplying the input vector by a predetermined connection weight matrix, the input vector including a product of a predetermined input weight matrix and the token and a product of a predetermined context weight matrix and the context information vector.

4. The information processing apparatus according to claim 3, wherein
in the output sequence generation process, the output sequence is further generated by multiplying the high-dimensional feature vector by an output weight matrix obtained by pre-training.

5. The information processing apparatus according to claim 4, wherein the at least one processor further carries out
a learning process for training the output weight matrix with reference to training data including a plurality of sets of (i) the medical sentence and the context information and (ii) a positive or negative label regarding a given word contained in the medical sentence.

6. The information processing apparatus according to claim 1, wherein
the context information is information indicative of a job title of a person who has written the medical sentence.

7. The information processing apparatus according to claim 1, wherein
the context information is a name of a field in which the medical sentence is recorded in the electronic medical record.

8. An information processing method comprising:
acquiring a token sequence obtained from a medical sentence in an electronic medical record and a context information vector obtained from context information of the electronic medical record; and
carrying out an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

9. The information processing method according to claim 8, wherein
the output sequence generation process includes
generating the token sequence by transforming each word contained in the medical sentence into a token by embedding the each word in a first vector space defined in advance, and
generating the context information vector by extracting predetermined context information from the electronic medical record and embedding the predetermined context information in a second vector space defined in advance.

10. The information processing method according to claim 9, wherein
in the output sequence generation process,
an input vector is further transformed into the high-dimensional feature vector by multiplying the input vector by a predetermined connection weight matrix, the input vector including a product of a predetermined input weight matrix and the token and a product of a predetermined context weight matrix and the context information vector.

11. The information processing method according to claim 8, wherein
in the output sequence generation process,
the output sequence is further generated by multiplying the high-dimensional feature vector by an output weight matrix obtained by pre-training.

12. The information processing method according to claim 11, further comprising
training the output weight matrix with reference to training data including a plurality of sets of (i) the medical sentence and the context information and (ii) a positive or negative label regarding a given word contained in the medical sentence.

13. The information processing method according to claim 8, wherein
the context information is information indicative of a job title of a person who has written the medical sentence.

14. The information processing method according to claim 8, wherein
the context information is a name of a field in which the medical sentence is recorded in the electronic medical record.

15. A non-transitory storage medium having a program stored therein, the program causing a computer to function as an information processing apparatus, the program causing the computer to carry out:
an acquisition process for acquiring a token sequence obtained from a medical sentence in an electronic medical record and a context information vector obtained from context information of the electronic medical record; and
an output sequence generation process for generating an output sequence from the token sequence and the context information vector, the output sequence generation process including a process for transformation into a high-dimensional feature vector that has a higher dimension than a sum of a dimension of the token sequence and a dimension of the context information vector.

16-21. (canceled)
